(19) United States
(12) Patent Application Publication
(10) Pub. No.: US 2004/0087576 A1
(43) Pub. Date: May 6, 2004

(54) Antimnemonic therapy for hypermemory syndromes

(76) Inventor: John L. Haracz, Petaluma, CA (US)

Correspondence Address: DAVID PRESSMAN, 1070 GREEN STREET, #1402, SAN FRANCISCO, CA, 94133-5418, US

(21) Appl. No.: 10/298,191

(22) Filed: Nov. 5, 2002

(51) Int. Cl. A61K 31/5513; A61K 31/46; A61K 31/485; A61K 31/445; A61K 31/416

(52) U.S. Cl. 514/221; 514/304; 514/282; 514/317; 514/406

(57) ABSTRACT

The present invention relates to antimnemonic therapy for the treatment of behaviroal disorders such as addiction, obsessive-compulsive disorder, Tourette's Syndrome, post-traumatic stress disorder (PTSD), bipolar disorder, depression, schizophrenia, anxiety disorders and personality disorders. Antimnemonic therapy may involve cue- or psychotherapy-induced reactivation of memories in combination with the administration of antimnemonic drugs.

### REFERENCE TO RELATED APPLICATIONS

This application claims the benefit of U.S. Provisional Application No. 60/332,713, filed Nov. 6, 2001.

### FIELD OF THE INVENTION

The present invention relates to treatment of behavioral disorders, particularly hypermemory disorders using antimnemonic therapy in combination with memory reactivation.

### BACKGROUND OF THE INVENTION

The pathophysiologies underlying many of the behavioral disorders listed in DSM-IV-TR (American Psychiatric Association, 2000) are unknown. The present invention proposes the reconceptualization of these disorders as hypermemory syndromes. Basic and clinical research findings suggest that overly entrenched memories, and corresponding entrenched neural plasticity underlying the memories, are associated with and may be central to the pathophysiology of these disorders. These hypermemory syndromes or disorders include, but are not limited to, disorders such as addictions, obsessive-compulsive disorder, Tourette's syndrome, post-traumatic stress disorder (PTSD), bipolar disorder, depression, schizophrenia, anxiety disorders and other disorders involving troubling memories. Reconceptualization of these disorders as hypermemory syndromes suggests new therapies using antimnemonic, or memory-impairing, drugs. One of the aims of the present invention is to reverse the entrenched memories underlying these disorders by activating the pathological memory and pharmacologically blocking its reconsolidation.

The pathological memories associated with a disorder are not necessarily conscious. Thus, people with one or more of these behavioral disorders may not complain of conscious, troubling memories. Research on the neuropsychology of memory (Eichenbaum & Cohen, From Conditioning to Conscious Recollection, New York: Oxford University Press, 2001) reveals that some types of memory are associated with conscious awareness (e.g., working memory and declarative memory), whereas other types of memory are largely unconscious (e.g., memories of habits or skills).

Memory impairing drugs, herein referred to as “antimnemonic drugs” have been used in basic research pertaining to the study of memory reconsolidation. This research suggests that recalled or “reactivated” memories go through a reconsolidation phase that may recapitulate brain mechanisms similar to the neural plasticity underlying consolidation during the original formation of these memories. (See Przybyslawski & Sara, Behav. Brain Res. 84:241-46 (1997); Roullet and Sara, Neural Plasticity 6:63-68 (1998); Przybyslawski et al., J. Neurosci. 19:6623-28 (1999); Nadel & Land, Nature Rev. Neurosci. 1:209-212 (2000); Nader et al., Nature 406:722-26 (2000a); Nader et al., Nature Rev. Neurosci. 1:216-19 (2000b); Sara, Learn. Mem. 7:73-84 (2000a); and Sara, Nature Rev. Neurosci. 1:212-213 (2000b).) For example, a rat fear-conditioning study showed that anisomycin, a protein-synthesis inhibitor known to block initial memory consolidation, blocked reconsolidation of reactivated memories. In this study, rats received a cue followed by an electric shock to induce fear conditioning. One or 14 days later the rats were presented with the cue, to which they demonstrated a fear response, and then were immediately treated with anisomycin. When the rats were presented with the cue a further twenty four hours later, they did not display the fear response. (Nader et al. (2000a), supra). Thus, anisomycin-treated rats appeared to “forget” their fear conditioning because they no longer associated the electric shock with the cue.

In similar studies, substances and treatments other than protein synthesis inhibitors were also shown to induce amnesia by blocking the reconsolidation of retrieved memories. These substances and treatments include electroconvulsive shock (Misanin et al., Science 160:554-55 (1968)); N-methyl-D-aspartate (NMDA) receptor antagonists, such as MK-801 (Przybyslawski & Sara, supra); β noradrenergic receptor antagonists, such as propranolol (Przybyslawski et al., supra) and AP5 (Sara (2000a), supra); hypothermia (Mactutus et al., Science 204:1319-20 (1979)); and hippocampal lesioning (Nadel & Land, supra). A hypothesis explaining the above results proposes that retrieved memories are in a labile state, susceptible to disruption by memory-impairing agents. (Reviewed, e.g., in Nadel & Land, supra; Sara (2000a), supra; and Sara (2000b), supra.)

However, some researchers have expressed skepticism toward the above hypothesis and its possible implications in the treatment of memory-dependent conditions or disorders. Cahill et al. (Trends Neurosci. 24:578-81 2001), report inconsistent results in the disruption of reactivated memories depending on laboratory and other experimental conditions. Cahill et al. also point out that memories disrupted by amnesic treatments can show spontaneous recovery. Further, Sara ((2000a), supra) discusses the recovery of memories after disruption of initial consolidation by presentation of “reminders” or further cues. This observation raises the possibility of the eventual recovery of memories after memory reactivation and disruption of reconsolidation by amnesic treatments. The present inventor proposes that inconsistency in the prior art (Cahill et al. (2001), supra) derives from the failure of these studies to employ chronic antmnemonic drug treatment. Although this chronic drug treatment may offer the best chance at blocking reconsolidation, studies reviewed above showed that single administrations of memory-impairing drugs can be sufficient to block reconsolidation (Przybyslawski and Sara (1997), supra; Przybyslawski et al. (1999), supra; Nader et al. (2000a), supra). Thus, the present invention also takes into account the possibility that acute antimnemonic drug treatment may be sufficient to block reconsolidation in some patients undergoing antimnemonic therapy.

Numerous memory-improving drugs are being developed to treat mild cognitive impairment, Alzheimer's disease, and other disorders involving impaired memory (U.S. Pat. No. 5,338,738; Staubli U, et al. Proc. Natl. Acad. Sci. USA 91:777-781, 1994; U.S. Pat. No. 5,556,847; Lynch G. Neurobiol. Learn. Mem. 70:82-100, 1998; WO 2000050447; WO 2001068137; Sun M K and Alkon D L. J. Pharmacol. Exp. Ther. 297:961-967, 2001). However, the prior art offers little encouragement for adapting drug-induced memory impairment as a treatment for behavioral disorders.

In a rare attempt at clinically inhibiting memory, Pitman et al. (Biol. Psychiatry 51:189-192, 2002) administered propranolol with the aim of preventing the onset of PTSD. This study was designed on the basis of preclinical research showing that post-training propranolol administration blocked the consolidation of emotion-related memory in rats (Cahill et al., 2000). Within 6 hours of experiencing an acute psychologically traumatic event, patients at risk for PTSD began a 10-day course of propranolol, 40 mg, or placebo four times daily. When assessed 3 months after the trauma, ratings of PTSD symptoms were slightly, but significantly, lower in propranolol-treated patients. Propranolol was not administered in association with memory reactivation as in the present invention, which provides a novel treatment for already established PTSD. The results of Pitman et al. (2002), supra, may have been more favorable if propranolol was administered in conjunction with cue-, psychotherapy-, or homework-induced memory reactivation as in the embodiments described below.

Behavioral disorders such as drug and alcohol addiction have been treated with very limited success using the “cue elicited craving paradigm.” The existing version of the cue-elicited craving paradigm involves presenting addicts with drug-related cues (e.g., videotapes, audiotapes, actors performing simulated drug administration rituals, pictures or slides of white powder, crack pipes, bar scenes, etc.) designed to elicit craving. This paradigm has aimed to habituate the craving response by repeatedly presenting these cues. This particular paradigm has at least three obvious disadvantages: 1) No olfactory cues: Clinical experience with drug addicts and alcoholics suggests that olfactory cues are particularly potent triggers of craving (e.g., a potent cue for crack addicts would be burning baking soda or other substances used to “cut” cocaine); 2) Small scope of cues: The cues typically presented in laboratory or clinical settings probably represent only a small subset of the many drug-related cues encountered by addicts in their everyday lives. 3) No antimnemonic drug treatment: Finally, and most importantly, current cue-elicited craving paradigms rarely have been combined with drug treatments designed to accelerate the habituation process and antimnemonic drugs have not been employed.

Repetitive transcranial magnetic stimulation (rTMS) was recently developed as a noninvasive method of altering the excitability of neuronal circuitry in the brain. Preliminary studies of patients with focal dystonia, epilepsy, PTSD, depression, or schizophrenia have revealed modest symptom reductions after rTMS treatment (Weiss et al., Amygdala plasticity: The neurobiological implications of kindling. In: The Amygdala: A Functional Analysis, 2^{nd} Ed. Aggleton J P, Ed. New York: Oxford University Press, 2000, pp. 155-194; Hoffman R E and Cavus I. Am. J. Psychiatry 159:1093-1102, 2002; McDonald W M, et al., Electroconvulsive therapy: Sixty years of progress and a comparison with transcranial magnetic stimulation and vagal nerve stimulation. In: Neuropsychopharmacology: The Fifth Generation of Progress. Davis K L, et al., Eds. Philadelphia: Lippincott Williams & Wilkins, 2002, pp. 1097-1108). rTMS might decrease excitability in neuronal pathways mediating the presently hypothesized entrenched memory consolidation underlying hypermemory disorders. However, it is not clear if these memory-related neuronal pathways can be selectively affected by rTMS administered from the scalp. Nader et al. (2000a, supra) proposed that a selective reversal of memory-related mechanisms mediated the anisomycin-induced inhibition of reconsolidation in the fear conditioning paradigm.

A number of behavioral disorders (e.g., anxiety disorders, borderline personality disorder, and drug or alcohol addiction) have been treated with varying degrees of success by repeatedly exposing patients to situations that elicit symptoms of these disorders (Barlow D H, Ed., Clinical Handbook of Psychological Disorders: A Step-by-Step Treatment Manual, 3^{rd} Ed. New York: Guilford Press, 2001). This exposure treatment aims to induce extinction of the tendency for patients to respond to these situations with increased symptoms. Animal models of anxiety disorders have been used to screen for drugs that promote the extinction of conditioned fear. For example, D-cycloserine, a drug that promotes NMDA receptor activity, was recently found to facilitate the extinction of conditioned fear (Davis M. Biol. Psychiatry 51:1S, 2002; Walker D L. J. Neurosci. 22:2343-2351, 2002). Based on this preclinical research, clinical trials have begun using D-cycloserine with the aim of promoting extinction during exposure therapy for anxiety disorders (Davis, supra). However, an obstacle for this treatment approach is that symptoms of anxiety disorders can show resistance to extinction (Poulton R, et al. Behav. Res. Ther. 39:29-43, 2001; Poulton R and Menzies R G. Res. Ther. 40:197-208, 2002). Furthermore, extinction is a learning process that masks, but does not erase, memories potentially involved in generating symptoms of behavioral disorders (Falls W A, Extinction: A review of theory and the evidence suggesting that memories are not erased with nonreinforcement. In: Learning and Behavior Therapy. O'Donohue, Ed. Boston: Allyn and Bacon, 1998, pp. 205-229).

Like other forms of learning, extinction is blocked by NMDA receptor antagonists (Falls, et al. J. Neurosci. 12:854-863, 1992), which is a class of antimnemonic drugs used in the present invention. Thus, a course of antimnemonic therapy described herein may tend to block extinction while producing the more important effect of erasing symptom-generating memories. The net effect would be reduced symptoms with no need for extinction because an effective erasure of symptom-related memories would leave nothing to be extinguished.

The art teaches away from the use of antimnemonic drugs to treat addiction, providing that “a memory trace produced by addiction cannot be ‘erased’ by medication or psychotherapy, but the patient can learn coping mechanisms . . . that permit a drug-free lifestyle.” (C. O'Brien, Pharmacotherapy of Addictive Disorders, Abstract, NIDA/NIH National Conference on Drug Addiction Treatment: From Research to Practice, Apr. 8-9, 1998).

Thus, there exists a need in the art for effective treatments for behavioral disorders, including hypermemory disorders. The present invention proposes the use of antimnemonic treatments in combination with cue presentation and/or memory reactivation, such as by psychotherapy, in order to satisfy this need.

### SUMMARY OF THE INVENTION

The present invention relates to the use of antimnemonic therapy for the treatment of behavioral disorders. Antimnemonic therapy as described herein involves bringing together memory-impairing drug treatments and memory reactivation by way of psychotherapy, homework, or an adaptation of the cue-elicited craving paradigm. The therapy may be administered acutely or as a chronic regimen.

In one aspect, the present invention provides a method of treating a behavioral disorder comprising presenting a cue associated with the disorder to a patient and administering an antimnemonic drug to the patient. This may be repeated as needed to alleviate symptoms of the disorder. The alleviation of symptoms may be measured by a clinician using standard techniques.

The cue is preferably at least one of a visual, olfactory, aural, tactile, or gustatory cue. It may be presented in a clinical environment or as part of the patient's natural environment outside of the clinic.

In one embodiment the behavioral disorder is a hypermemory disorder. The hypermemory disorder may be selected from the group consisting of addiction, obsessive-compulsive disorder, Tourette's Syndrome, post-traumatic stress disorder (PTSD), bipolar disorder, depression, schizophrenia, anxiety disorders and personality disorders. In a preferred embodiment the hypermemory disorder is addiction.

In one embodiment the antimnemonic drug is selected from the group consisting of benzodiazepines, NMDA-receptor antagonists, dopamine receptor blockers, glucocorticoid receptor antagonists, α2-adrenoceptor agonists, β-adrenoceptor antagonists, muscarinic cholinergic antagonists, protein kinase A inhibitors, protein kinase C inhibitors, calcium/calmodulin dependent kinase inhibitors, mitogen-activated protein kinase kinase inhibitors, cyclic adenosine monophosphate response element binding protein inhibitors, nitric oxide synthase inhibitors and GABA receptor agonists.

In a preferred embodiment the antimnemonic drug is selected from the group consisting of memantine, muscimol, clonidine, metoprolol, atropine, ecopipam, sulpiride, haloperidol, 7-nitroindazole, benztropine, scopolamine, propranolol, dextromethorphan, midazolam and lorazepam.

In another embodiment the antimnemonic drug is memantine. The memantine is preferably administered at a dose of between 0.1 and 5 mg/day.

The antimnemonic drug may be administered by a clinician. Alternatively, the antimnemonic drug may be self-administered by the patient.

The antimnemonic drug is typically administered within five hours following cue presentation. In one embodiment the antimnemonic drug is administered within five minutes following cue presentation. However, in an alternative embodiment the antimnemonic drug is administered up to one hour prior to cue presentation.

In another aspect, a method of treating a behavioral disorder is provided comprising reactivating a memory associated with the disorder in a patient and administering an antimnemonic drug to the patient. These steps may be repeated as needed to alleviate symptoms or to maintain symptoms in an alleviated state.

In one embodiment reactivation is triggered by exposure of the patient to a cue. The cue is preferably at least one of a visual, olfactory, aural, tactile, or gustatory cue.

In another embodiment reactivation is triggered by psychotherapy. In a further embodiment reactivation is triggered by “field trips” to environments with cues that tend to elicit symptoms of hypermemory disorders. In yet another embodiment reactivation is triggered by homework involving the patient voluntarily recalling troubling memories or feelings. Reactivation may occur in a clinical or a non-clinical environment.

In one embodiment the behavioral disorder is a hypermemory disorder. In another embodiment the hypermemory disorder is a behavioral disorder included in the reward deficiency syndrome, preferably addiction.

In a further aspect, a method of treating addiction is provided. A patient diagnose as suffering from addiction is exposed to a cue associated with the addiction and an antimnemonic drug is administered.

In one embodiment the addiction is selected from the group consisting of addiction to drugs, gambling, food, sex, thrill-seeking, violence, political power, money and computer technology. In a preferred embodiment the addiction is addiction to drugs.

The drugs may be selected from the group consisting of alcohol, cocaine, nicotine, Cannabis, opiates and opiate derivatives.

When the patient is to be treated for addiction to drugs, the cue presentation may comprise exposure to audiotapes of drug-related stimuli, videotapes of drug-related stimuli, actors performing simulated drug administration rituals, drug paraphernalia or photographs of drug paraphernalia. Drug paraphernalia includes, for example, razor blades, needles, syringes, lighters and crack pipes.

When the addiction is nicotine addiction, the cue may comprise exposure to cigarette smoke. Similarly, when the addiction is addiction to alcohol, the cue may comprise exposure to the smell of alcoholic beverages and when the addiction is cocaine addiction, the cue may comprise exposure to the smell of burning baking soda or other substances used to “cut” or dilute cocaine.

### DETAILED DESCRIPTION OF THE PREFERRED EMBODIMENT

Definitions

“Addiction” is used broadly herein and refers to a syndrome characterized by compulsive behavior that results in an impairment in social and/or psychological functions and/or damage to health. For a definition of addiction in the context of drug use, see, e.g., O'Brien (Science 278:66-70 1997). The compulsive behavior is oriented to, for example, drugs, alcohol, gambling, food, sex, thrill-seeking, computer technology, and the like. These examples are illustrative and not limiting.

“Alleviation of symptoms,” in the context of a behavioral disorder, refers to improvement in the social or psychological function or health of a patient, as evaluated by any measure accepted in the art. Preferably, “alleviation of symptoms” is a clinically recognizable decrease in symptoms described in DSM-IV-TR (American Psychiatric Association, 2000) for particular disorders herein reconceptualized as hypermemory disorders. The psychosocial function of a patient may be evaluated using standard measures provided in DSM-IV-TR (American Psychiatric Association, 2001), such as the Global Assessment of Functioning Scale and the Social and Occupational Functioning Assessment Scale.

“Antimnemonic drug” refers broadly to any drug, compound, polypeptide or non-pharmaceutical treatment, such as gene therapy, that can be used to impair memory formation in a patient. The antimnemonic activity of a particular compound or treatment may be measured by any method known in the art, for example by administering standard neuropsychological tests of memory in animals or, more preferably, in human subjects (Lezak M D, Neuropsychological Assessement, 3^{rd} Ed. New York: Oxford University Press, 1995; Spreen 0 and Strauss E, A Compendium of Neuropsychological Tests: Administration, Norms, and Commentary, 2^{nd} Ed. New York: Oxford University Press, 1998; Curran H V, Psychopharmacological perspectives on memory. In: The Oxford Handbook of Memory. Tulving E and Craik F I M, Eds. New York: Oxford University Press, 2000, pp. 539-554, each of which is expressly incorporated herein in its entirety). This clinical research uses a number of methods to detect selective drug effects on memory distinct from effects on arousal or attention (Curran H V, supra). Clinical tests of memory include, but are not limited to, the Wechsler Memory Scale-Revised, California Verbal Learning Test, word-list recall, continous word recognition, word stem or fragment completion, serial reaction time, and the Tower of Hanoi. Drug-induced memory impairment may be detected by testing nonverbal memory in animals (Curran H V, supra). Preclinical tests of memory include, but are not limited to, discrimination learning, fear conditioning, active or passive avoidance, the Morris water maze, delayed matching to position, delayed nonmatching to sample, and conditioned taste aversion. An antimnemonic drug may have other activities in addition to its activity in memory impairment. Thus, a drug or treatment with indications unrelated to memory is considered an antimnemonic drug if it has memory-impairing activity, even if that activity is widely considered to be a side effect.

Antimnemonic drugs include, but are not limited to, dopamine receptor blockers, NMDA receptor antagonists (e.g., memantine [WO0245710], dextromethorphan [U.S. Pat. No. 6,207,674], and N20C [Planells-Cases R et al. J. Pharmacol. Exp. Ther. 302:163-173, 2002.]); benzodiazepines (e.g., midazolam [Rammsayer T H, et al. Cognitive Brain Res. 9:61-71, 2000], alprazolam, triazolam, diazepam, and lorazepam [Krystal J H, et al. Psychopharmacology 135:213-229, 1998; Curran H V, supra]), gamma-aminobutyric acid (GABA) receptor agonists (e.g., muscimol [Castellano C and McGaugh J L. Behav. Neural Biol. 54:156-164, 1990]), glucocorticoid receptor antagonists, α2-adrenoceptor agonists (e.g., clonidine [McGaugh J L, et al., Amygdala: Role in modulation of memory storage. In: The Amygdala: A Functional Analysis, 2^{nd} Ed. Aggleton J P, Ed. New York: Oxford University Press, 2000, pp. 391-423]), β-adrenoceptor antagonists (e.g., propranolol [McGaugh J L, et al. 2000, supra] and metoprolol [Cahill L, et al. Neurobiol. Learn. Mem. 74:259-266, 2000]), muscarinic cholinergic antagonists (e.g., atropine [Gasbarri A, et al. Brain Res. 627:72-78, 1993], benztropine and scopolamine [Curran H V, supra; Rammsayer T H, et al. Cognitive Brain Res. 9:61-71, 2000]), D1-like dopamine receptor antagonists (e.g., SCH 23390 [Castellano C, et al. Behav. Neural Biol. 56:283-291, 1991] and ecopipam [Romach M K, et al. Arch. Gen. Psychiatry 56:1101-1106, 1999]), D2-like dopamine receptor antagonists (e.g., sulpiride [Castellano C, et al. Behav. Neural Biol. 56:283-291, 1991; Gasbarri A, et al. Brain Res. 627:72-78, 1993]), nonselective dopamine receptor antagonists (e.g., haloperidol [Rammsayer T H, et al. Cognitive Brain Res. 9:61-71, 2000]), protein kinase A inhibitors, protein kinase C inhibitors, calcium/calmodulin-dependent kinase inhibitors, mitogen-activated protein kinase kinase inhibitors (e.g., U0126 [Schafe G E, et al. J. Neurosci. 20:8177-8187, 2000]), cyclic adenosine monophosphate response element binding protein inhibitors, and nitric oxide synthase inhibitors (e.g., N-omega-nitro-L-arginine methyl ester and 7-nitro indazole [Haracz J L, et al. Brain Res. 746:183-189, 1997]).

“Chronic,” in the context of the treatment of a behavioral disorder, particularly a hypermemory disorder, refers to therapy that is administered on more than one occasion. Chronic antimnemonic therapy preferably involves the continuation of regular therapeutic sessions as long as the patient continues to suffer from a behavioral disorder. “Acute,” in the context of the treatment, refers to therapy administered on a single occasion.

A “cue” refers to any stimulus that can be perceived by one or more of the human senses (e.g., a visual, olfactory, aural, tactile, or gustatory stimulus). Preferably the cue is one that has some associative significance and thus triggers a particular response by the perceiving individual, such as memory reactivation in a subject with one or more hypermemory disorders. As used herein, the term “cues” includes verbal cues.

In one embodiment a cue is an object, smell, sound, picture or other stimulus that tends to trigger symptoms of a specific behavioral disorder. In a preferred embodiment of the present invention, a cue elicits a memory in a patient. Preferably the memory is associated with a behavioral disorder from which the patient is suffering, such as a hypermemory disorder. As a component of antimnemonic therapy, a patient suffering from a behavioral disorder, particularly a hypermemory disorder, may be intentionally exposed to one or more cues in a clinical setting or in a non-clinical environment. An example of the latter may include a “field trip” intentionally taken to expose a patient to the broad scope of cues she encounters in the patient's everyday life. Exposure of a patient to a cue may also occur through a patient's chance encounter with a cue in a non-clinical environment. In addition, exposure to a cue may occur outside of the clinical setting when a patient completes “homework” assigned by the clinician. “Homework” as used herein, comprises instructions for the patient to focus on memories or feelings associated with their disorder outside of the clinic in homework sessions that are approximately equal in duration to psychotherapy sessions.

“Hypermemory syndrome,” also called “hypermemory disorder” refers to a broad category of disorders, particularly behavioral disorders, that are herein hypothesized to involve entrenched memories and entrenched neural plasticity underlying the memories. Examples of hypermemory disorders include, but are not limited to, those disorders that have been classified as “reward deficiency syndrome;” (See, e.g., Blum, U.S. Pat. No. 6,132,724; and Blum et al., “Reward Deficiency Syndrome,” (American Scientist, March-April 1996), both of which are incorporated herein by reference in their entirety).

Hypermemory disorders include, without limitation, addictions, e.g., to drugs (such as, cocaine, nicotine, Cannabis, opiates and opiate derivatives, and the like), alcohol, gambling, food, sex, thrill-seeking, computer technology, etc.; obsessive-compulsive disorder; Tourette's Syndrome; post-traumatic stress disorder (PTSD); bipolar disorder; depression; schizophrenia; anxiety disorders, including panic and phobias; personality disorders, including antisocial personality disorder; and other disorders involving troubling memories. For the purposes of the present invention, the “worried well” (i.e., people who seek therapy despite not having a specific neuropsychiatric diagnosis) are also considered to suffer from hypermemory disorders. Individuals would also be considered to suffer from a hypermemory disorder if they complain of maladaptive lifestyles dominated by troubling memories. The above disorders are considered hypermemory disorders even if the patient and/or a clinician evaluating and treating the patient are unaware of specific memories that are associated with the disorder.

A “memory associated with a disorder” refers to a memory that is linked causally or simply by association to a disorder. In one embodiment the memory is associated with a behavioral disorder. In another embodiment the memory is associated with a hypermemory disorder. For example, the memory of a traumatic experience is a memory that may be associated with a hypermemory disorder, particularly post traumatic stress disorder (PTSD). The memories of feelings or experiences associated with prior alcohol or drug use are also memories associated with a hypermemory disorder, particularly addiction. A memory associated with a disorder may be a conscious or unconscious memory.

“Psychotherapy” refers broadly to forms of psychiatric treatment which employ specialized communication techniques practiced by a properly trained physician, counselor, or clinician for the purpose of curing or reducing or alleviating a behavioral disorder of a patient and improving the patient's emotional, social, and/or mental health. As a component of antimnemonic therapy, psychotherapy is administered with the goal of helping the subject experience the reactivation of memories or feelings associated with the subjects behavioral disorder. For this purpose, the psychotherapist preferably directs the subjects to focus on troubling memories or feelings associated with the subject's disorder.

“Reactivation of a memory” refers to a subject's recollection of a memory associated with a disorder. Reactivation may occur either spontaneously or be associated with the presentation of one or more stimuli or cues that elicit the memory. For example, a memory associated with nicotine or alcohol addiction may be reactivated by the site or smell of a burning cigarette or an alcoholic beverage, respectively. Reactivation of a memory can manifest in a patient by a behavior or feeling. For example, a patient suffering from addiction may experience craving when undergoing memory reactivation. In a psychotherapy session or in a session in which cues are presented to a patient, “reactivation” may be considered to take place at any point during the session, as may be demonstrated by the patient's subjective report and/or objective measures such as elevations in heart rate or blood pressure. Reactivation of a memory does necessarily result in conscious recollection of a memory. Reactivation may occur in a clinical environment, as when a clinician directs the patient to focus on troubling memories or feelings, or may occur in a non-clinical setting, as when a patient focuses on troubling memories or feelings in homework sessions, or when a patient encounters relevant cues during “field trips” or in their everyday lives.

“Reward deficiency syndrome” refers to a category of disorders, particularly neuropsychiatric disorders, including, without limitation, addictive behaviors (alcoholism, substance abuse, smoking, and obesity); impulsive behavior, including attention-deficit disorder (ADD), Tourette's syndrome, anxiety disorders, and personality disorders (e.g., conduct disorder and antisocial personality disorder). See, e.g., Blum et al., “Reward Deficiency Syndrome,” 1996, supra, and U.S. Pat. No. 6,132,724, supra, expressly incorporated by reference herein.

Treatment of Disorders Using Antimnemonic Therapy

In one aspect, antimnemonic therapy generally involves administering memory-impairing drugs combined with methods that are individually tailored for eliciting specific memories from people who suffer from a particular behavioral disorder. These methods include, without limitation, cue presentations, psychotherapy, field trips and homework.

Memory consolidation involves numerous neurotransmitters, hormones, and intracellular signaling pathways (Gasbarri A, et al. Brain Res. 627:72-78, 1993; Cahill L and McGaugh J L. Trends Neurosci. 21:294-299, 1998; Silva A J, et al. Annu. Rev. Neurosci. 21:127-148, 1998; Sara S J, et al. Learn. Mem. 6:88-96, 1999; Berman D E, et al. J. Neurosci. 20:7017-7023, 2000; Abel T and Lattal K M. Curr. Opin. Neurobiol. 11:180-187, 2001; Blair H T, et al. Learn. Mem. 8:229-242, 2001; Kandel E R. Science 294:1030-1038, 2001; McGaugh J L and Roozendaal B. Curr. Opin. Neurobiol. 12:205-210, 2002). A variety of drugs, including many that have been well-established in use in treatment methods other than antimnemonic therapy, are known to influence memory formation. Many of these drugs have known memory-impairing functions and, therefore, are useful in antimnemonic therapy. Traditionally, the memory-impairing functions of these drugs have been viewed as negative, undesirable side effects. Antimnemonic therapy uniquely enables exploitation of these memory-impairing functions for therapeutic benefit. Preferred drugs for use in antimnemonic therapy are drugs that have been found to be safe in their use in other contexts and have demonstrable memory-impairing effects. However, any antimnemonic drug or treatment may be used in antimnemonic therapy with the proper clinical supervision.

Antimnemonic drugs that may be used for antimnemonic therapy include, but are not limited to, NMDA receptor antagonists (e.g., memantine [WO0245710], dextromethorphan [U.S. Pat. No. 6,207,674], and N20C [Planells-Cases R et al. J. Pharmacol. Exp. Ther. 302:163-173, 2002.]), benzodiazepines (e.g., midazolam [Rammsayer T H, et al. Cognitive Brain Res. 9:61-71, 2000], alprazolam, triazolam, diazepam, and lorazepam [Krystal J H, et al. Psychopharmacology 135:213-229, 1998; Curran H V, Psychopharmacological perspectives on memory. In: The Oxford Handbook of Memory. Tulving E and Craik F I M, Eds. New York: Oxford University Press, 2000, pp. 539-554]), gamma-aminobutyric acid(A) receptor agonists (e.g., muscimol [Castellano C and McGaugh J L Behav. Neural Biol. 54:156-164, 1990]), glucocorticoid receptor antagonists, α2-adrenoceptor agonists (e.g., clonidine [McGaugh J L, et al., Amygdala: Role in modulation of memory storage. In: The Amygdala: A Functional Analysis, 2^{nd} Ed. Aggleton J P, Ed. New York: Oxford University Press, 2000, pp. 391-423]), β-adrenoceptor antagonists (e.g., propranolol [McGaugh J L, et al., Amygdala: Role in modulation of memory storage. In: The Amygdala: A Functional Analysis, 2^{nd} Ed. Aggleton J P, Ed. New York: Oxford University Press, 2000, pp. 391-423] and metoprolol [Cahill L, et al. Neurobiol. Learn. Mem. 74:259-266, 2000]), muscarinic cholinergic antagonists (e.g., atropine [Gasbarri A, et al. Brain Res. 627:72-78, 1993], benztropine and scopolamine [Curran H V, supra; Rammsayer T H, et al. Cognitive Brain Res. 9:61-71, 2000]), D1-like dopamine receptor antagonists (e.g., SCH 23390 [Castellano C, et al. Behav. Neural Biol. 56:283-291, 1991] and ecopipam [Romach et al., 1999, supra]), D2-like dopamine receptor antagonists (e.g., sulpiride [Castellano C, et al., supra; Gasbarri A, et al. Brain Res. 627:72-78, 1993]), nonselective dopamine receptor antagonists (e.g., haloperidol [Rammsayer T H, et al., 2000, supra]), protein kinase A inhibitors, protein kinase C inhibitors, calcium/calmodulin-dependent kinase inhibitors, mitogen-activated protein kinase kinase inhibitors (e.g., U0126 [Schafe G E, et al., supra]), cyclic adenosine monophosphate response element binding protein inhibitors, and nitric oxide synthase inhibitors (e.g., N-omega-nitro-L-arginine methyl ester and 7-nitro indazole [Haracz J L, et al. Brain Res. 746:183-189, 1997]).

Antimnemonic therapy as described herein may be used to treat any behavioral disorder, including neuropsychiatric disorders, because troubling conscious or unconscious memories may have at least an adverse shaping influence on some behaviors and symptoms associated with any behavioral disorder. Antimnemonic therapy may also be used to treat behavioral disorders that are not clinically classified as neuropsychiatric disorders, but that are characterized by a maladaptive behavior associated with one or more memories, particularly troubling memories. In one embodiment the patient to be treated is diagnosed as suffering from a hypermemory disorder, more preferably a hypermemory disorder selected from the group consisting of addictions (e.g., to drugs [such as cocaine, nicotine, Cannabis, opiates and opiate derivatives, and the like], alcohol, gambling, food, sex, thrill-seeking, violence, political power, money, computer technology, etc. . . ); obsessive-compulsive disorder; Tourette's Syndrome; post-traumatic stress disorder (PTSD); bipolar disorder; depression; schizophrenia; anxiety disorders; personality disorders (e.g., antisocial personality disorder [APD]) and other disorders involving troubling memories.

In one embodiment the behavioral disorder amenable to antimnemonic therapy is a hypermemory disorder other than an anxiety disorder. In another embodiment the behavioral disorder amenable to antimnemonic therapy is a hypermemory disorder other than PTSD.

A patient is preferably diagnosed as suffering from a disorder amenable to treatment with antimnemonic therapy by a clinician, preferably a psychiatrist or psychologist.

In a preferred embodiment, antimnemonic therapy involves administration of memory-impairing drugs combined with presentation of cues related to the disorder. Preferably the cues are individually tailored for eliciting memories associated with the particular disorder from which the patient suffers. However, it is not necessary for a patient to be consciously aware of the reactivation of a memory.

The cues may be presented in a clinical setting, where they are preferably prepared by the clinician. The cues may take any form. For example, without limitation, the cues may be pictures, slides, videotapes, films, audiotapes, objects, smells, music or spoken dialog, including acting. The cues may be chosen by the clinician based on general knowledge regarding the specific disorder from which the patient is suffering. Preferably, however, the cues are chosen by the clinician based on discussion with the patient regarding the disorder so that cues with particular relevance to the patient are used. In another embodiment the cues are “natural” and are not prepared by the clinician. The patient may be exposed to these cues in the course of everyday activity. Alternatively, the patient may be exposed to ‘natural’ cues in an arranged visit or “field trip” to a specific location chosen based on the likelihood of encountering relevant cues. In a further embodiment, the patient is given “homework,” such that the relevant cues may be presented at the patients discretion in the surroundings chosen by the patient, such as in the patient's home.

In a further embodiment, a conscious memory associated with the disease or disorder is elicited. Memory reactivation is combined with administration of the antimnemonic drug. The method or methods by which a memory is elicited is not limited in any way. The memory may be elicited by the presentation of cues, as described above. In one embodiment the memory is elicited by psychotherapy individually tailored for eliciting memories associated with the disorder. In other embodiments meditation or verbal suggestion is used to elicit a memory associated with the disorder. In still other embodiments the memory is elicited outside of the clinical setting by cues encountered by the patient in daily life or as a result of “homework” assigned by the clinician.

In a particular embodiment, treatment of addiction utilizes cue-induced elicitation of memories by presenting the patient with olfactory cues. For example, patients suffering from addiction to nicotine, alcohol or cocaine may presented with smells generated by cigarette smoke, alcoholic beverages, or the burning of baking soda and other substances used to “cut” cocaine, respectively. In other embodiments, addicts are presented with photographs or videotapes of objects associated with the addiction, such as drug paraphernalia. In still other embodiments, addicts are presented with audiotapes of drug related activity, or actors performing simulated drug administration rituals.

In another embodiment of the invention, treatment of addiction encompasses exposure of addicts to a broad scope of cues, rather than a single cue. For example, a patient may be placed in a situation, either in the clinic or in a natural setting, in which he or she will encounter a wide variety of cues associated with the disorder from which they are suffering. For example a patient suffering from gambling addiction may take a field trip to a casino.

Cue presentation and/or memory reactivation is combined with administration of one or more antimnemonic drugs. Although discussed below in terms of cue presentation, the discussion is also applicable to the conscious memory reactivation embodiment.

The antimnemonic drug is typically administered following cue exposure. However, it may be administered prior to cue presentation. For example, depending on the particular pharmacokinetics of the drug that is used, the drug may be administered prior to cue presentation so that it reaches peak efficacy at a desired point following cue presentation.

In one embodiment at least one antimnemonic drug is administered by the clinician up to one hour prior to cue exposure. In another embodiment at least one antimnemonic drug is self-administered by the patient prior to cue exposure in a natural setting, or prior to a “homework” session involving mentally focusing on troubling memories or feelings.

The antimnemonic drug is preferably administered while the cue and/or elicited memory is still fresh in the patients mind, preferably up to five hours following presentation of the cue. More preferably the antimnemonic drug is administered within 2 hours following cue exposure, yet more preferably within one hour, still more preferably within a half hour and even more preferably within fifteen minutes following cue exposure. In the most preferred embodiment of the present invention, the antimnemonic drug is administered within thirty minutes after cue presentation.

The antimnemonic drug may be administered either singly or as a drug cocktail comprising two or more drugs, Such as two drugs from different pharmacological classes with differing mechanisms of action. For example, a combination of an NMDA blocker and a benzodiazepine may be used. The drug-cocktail embodiment is based on preclinical and clinical results showing that some drug combinations are particularly effective in disrupting learning and memory (See Anglade F, et al. J. Physiol., Paris 93:225-232, 1999; Cain et al., Behav. Brain Res. 111:125-37, 2000; Krystal, 1998, supra; Smith-Roe and Kelley, 2000, J. Neurosci. 20:7737-7742, 2000).

The antimnemonic drug is chosen on the basis of effectiveness at inhibiting memory while having the fewest undesirable side effects. It is contemplated that a variety of different drugs and drug combinations may be employed over the course of treatment to determine which drug or combination is most effective for a patient suffering from a particular disorder.

Drug dosages and the routes of administration are the same as those familiar to clinicians skilled in the art and experienced with administering these drugs according to treatment methods other than antimnemonic therapy. Exemplary dosages and methods of administration may be found, for example, in the Physicians Desk Reference ( ). The clinician will adjust the dosages to achieve the desired affect while avoiding undesirable side effects. Preferably the lowest effective dosage will be used. Such determinations are within the level of skill in the art for the ordinary clinician. For example, NMDA receptor antagonists such as memantine are useful in antimnemonic therapy because drugs of this class impair memory in humans (Krystal J H, et al. Psychopharmacology 135:213-229, 1998) and animals (McGaugh J L, et al., 2000, supra). In studies of patients with neuropsychiatric disorders involving neurodegeneration, memantine was administered orally in a dosage range from 0.1 to 1000 mg/day (WO0245710). In this dosage range, memantine specifically acts as a noncompetitive NMDA receptor blocker.

Although memantine is well tolerated in patients at doses of 100-500 mg/day, clinicians administer a preferred therapeutic dose of 5-35 mg/day. In a preferred embodiment, a memantine dose of 0.1-5 mg/day is used in antimnemonic therapy as described herein. This dose is sufficient to block NMDA receptors, but is low enough to minimize side effects. Combined low doses of drugs from different pharmacological classes can dramatically impair memory in humans while minimizing side effects such as sedation (Krystal JH, et al. Psychopharmacology 135:213-229, 1998). Within the memantine dose range of the preferred embodiment (0.1-5 mg/kg), a clinician can adjust the patient's dose upward or downward as needed to achieve a therapeutic effect while minimizing side effects.

The specific dose level of an antimnemonic drug for any particular patient may depend on a variety of factors including the patient's age, body weight, general health, sex, rate of excretion, drug combination, and the severity of the particular hypermemory disorder undergoing treatment. The following examples of treatment schedules for specific hypermemory disorders are illustrative and do not limit the invention.

The antimnemonic drugs may be administered by the clinician in a clinical setting. However, in the case where the cues are those provided in the patients everyday life, the patient may self-administer the drugs following exposure to the appropriate cues.

For subjects with any of the hypermemory syndromes described above, antimnemonic drugs may be regularly taken 1-3 times per day on days without psychotherapy or cue-exposure sessions, particularly if these subjects often dwell upon troubling memories.

As described above, in a typical session of antimnemonic therapy a patient is exposed to a cue, followed by administration of an antimnemonic drug. A single cue may be used, or a series of cues may be presented together. The length of time a patient is exposed to a cue may depend upon the nature of the cue and the relevance of the cue as reported by the patient. In a preferred embodiment, a patient in a clinical setting is exposed to cues for 10 minutes to 2 hours, more preferably for from 30 minutes to 60 minutes. The actual time of cue exposure will be determined by the clinician based on the nature of the cues. This procedure may be repeated two or more times in a single day, depending on a number of factors including the nature of the cues, the type of antimnemonic drug being employed and the tolerance of the patient for the treatment.

Antimnemonic therapy may be “acute” and consist of a single cue exposure session combined with administration of an antimnemonic drug. Typically, antimnemonic therapy will be chronic and a number of antimnemonic therapy sessions will be provided as necessary to effectively treat the disorder. Thus, the antimnemonic therapy is preferably administered as necessary to alleviate symptoms and/or to maintain symptoms in an alleviated state. The effectiveness of the antimnemonic therapy may be assessed by the clinician according to standard methods. Alternatively, the effectiveness of the therapy may be determined based on the patients perception of any change in the symptoms of the disorder. The sessions may be repeated as often as deemed necessary by the physician to control the disorder, or as often as desired by the patient to control undesired symptoms. The frequency of sessions may be limited by the nature of the cues, the type of drug being employed and/or the tolerance of the patient for the therapy. In one embodiment the therapy is repeated every day. In other embodiments the therapy is repeated at least once a week, at least once a month and at least once a year. In a further embodiment the therapy is repeated whenever the patient complains of undesirable symptoms of the disorder.

Patients may be treated with other types of therapy concurrent with antimnemonic therapy. For example, and without limitation, patients suffering from depression may be concurrently treated with known anti-depressant medications, patients suffering from schizophrenia may be concurrently treated with anti-psychotics and patients suffering from bipolar disorder may be concurrently treated with lithium.

In addition, antimnemonic therapy may be combined with meditation. Meditation is included only if the treatment recipient is comfortable with it and is willing and able to follow meditation instructions. For meditation, the recipient is instructed to sit in a quiet environment for up to one hour while avoiding any dwelling upon memories or other thoughts. Alternatively, meditation can last for time periods substantially different from one hour depending on the recipient's comfort with the procedure. Antimnemonic drugs may be administered in combination with the meditation.

The following examples of treatment schedules for specific hypermemory syndromes are illustrative and do not limit the invention in any way. All references cited herein are hereby incorporated by reference.

### EXAMPLES

A) Addictions

A patient diagnosed as suffering from an addiction is treated with antimnemonic therapy. A number of theories of drug addiction suggest that the addiction is learning based (Stewart J, et al. Psychological Rev. 91:251-268, 1984; Robinson T E and Berridge K C. Brain Res. Rev. 18:247-291, 1993; White N M. Addiction 91:921-949, 1996; O'Brien C P, et al. J. Psychopharmacol. 12:15-22, 1998; Di Chiara G. Eur. J. Pharmacol. 375:13-30, 1999; Haracz J L, et al. Ann. N.Y. Acad. Sci. 877:811-819, 1999; Robbins T W and Everitt B J. Nature 398:567-570, 1999; Hyman S E and Malenka R C. Nature Rev. Neurosci. 2:695-703, 2001). Thus, administration of antimnemonic drugs along with cue-elicited reactivation of craving may be useful in erasing memories that compel addicts toward the focus of their addiction.

After consultation with the patient, a clinician selects cues relevant to the patients addiction. The cues are presented to the patient in clinical cue-exposure sessions lasting approximately 30-60 minutes per day. If the nature of the cue permits, the cue-exposure sessions can last for a time period substantially different from 30-60 minutes depending on the addict's comfort with this procedure. The cue exposure sessions preferably comprise the presentation of visual cues relevant to the patient's specific addiction. Together with visual-cue presentation, addicts may also be presented with olfactory and auditory cues. For example, cocaine addicts may be presented with the smell of burning baking soda or other substances commonly used to cut cocaine, while gambling addicts may be presented with the sounds of a casino. The cue-exposure sessions may be repeated daily, several times a week, or less often than once weekly depending, in part, on the willingness and ability of the treatment recipient to commit time to treatment, the type of antimnemonic drugs administered and the therapeutic effectiveness of the treatments. In addition, the type of antimnemonic drug administered and the content of the cues may be varied from session to session to find the optimal therapeutically effective combination.

Immediately after each cue presentation session, one or more antimnemonic drugs are administered in the absence of cues. Alternatively, the antimnemonic drug may be administered prior to the cue presentation session, as described above. The addict preferably sits quietly or engages in light physical exercise for approximately two hours following drug administration.

Once the addict reports consistently decreased subjective craving, anxiety, or excitement during cue-exposure sessions, the sessions are replaced with a similar schedule of trips to environments outside of the clinic that provide relevant cues. Exposure to the environments is similarly followed by antimnemonic drug administration. These field trips typically involve 30-60 minute exposures to the non-clinical environments relevant to the specific addictions. Alternatively, the exposures can last for time periods substantially different from 30-60 minutes depending on the addict's comfort with this procedure.

The number of field trips and clinical sessions is decreased or discontinued once the addict reports consistently decreased subjective responses to the environments. However, if the addict continues to experience cravings after this treatment regimen, or cravings return after the number of sessions is decreased or discontinued, the therapy may be reinitiated.

In another embodiment, antimnemonic drugs are prescribed to be taken 1-3 times daily as needed immediately after the onset of craving, even if there is no explicit or recognized cue presentation.

Concurrent with or after any phase of the treatment regimen, depending on the willingness and ability of the addict, meditation is prescribed once daily. Antimnemonic drugs are prescribed to be taken as needed prior to or after meditation if the addict experiences craving during meditation.

B) Obsessive-Compulsive Disorder and Tourette's Syndrome

A patient is diagnosed as suffering from obsessive-compulsive disorder or Tourette's Syndrome. In consultation with a clinician, the patient reports that symptoms are typically elicited by specific environmental stimuli. The clinician then selects cues for presentation in clinical cue-exposure sessions as described above. Following cue exposure, the patient is treated with an antimnemonic drug. Alternatively, the patient is prescribed the antimnemonic drug and instructed to self-administer the drug after exposure to the relevant cues during the course of his everyday activities or after the onset of symptoms. In a further alternative, the patient is intentionally placed in a non-clinical environment where he is likely to be exposed to relevant cues. Following exposure the antimnemonic drug is administered. This drug treatment may prevent the reconsolidation of implicit, habit-forming memories underlying the disorders (Leckman et al., Chinese Med. J. 64:669-692 2001).

C) Post-Traumatic Stress Disorder (PTSD), Bipolar Disorder, and Other Disorders Involving Anxiety or Troubling Memories

A patient diagnosed with PTSD, bipolar disorder or suffering from another disorder involving anxiety or troubling memories reports symptoms that are typically elicited by specific environmental cues. The patient is treated with antimnemonic therapy as described above for obsessive and compulsive disorder and Tourette's syndrome. However, if the patient's symptoms are more related to intrusive troubling memories, than the cue-exposure sessions and environmental cue exposure are replaced with psychotherapy sessions or discussion designed to elicit these memories. Antimnemonic drugs are taken immediately after these psychotherapy sessions or after spontaneous symptom onset. During the psychotherapy sessions, subjects are preferably instructed to focus on troubling memories or sources of anxiety for approximately 30-60 minutes. Alternatively, this focus may be maintained for time periods substantially different from 30-60 minutes depending on the subject's comfort with this procedure. Meditation may be prescribed depending on the willingness and ability of the subject. Patients may also undertake homework sessions that are similar to psychotherapy sessions in duration and purpose, in which the patient focuses on troubling memories or sources of anxiety. Preferably, homework sessions are carried out on days without cue or psychotherapy sessions. Antimnemonic drugs are self-administered prior to or after the homework sessions.

Concurrent with this antimnemonic therapy regimen, subjects with bipolar disorder may take a traditional drug treatment, such as lithium, familiar to clinicians skilled in the art. Antimnemonic therapy may enable a tapering off or dose-reduction of traditional drug treatment.

D) Depression

Depressed people may often ruminate on troubling memories (Schacter, D. L. The Seven Sins of Memory. Boston: Houghton Mifflin 2001). In these cases, antimnemonic therapy may usefully treat symptoms of depression. Patients diagnosed as suffering from depression and reporting troubling memories preferably receive antimnemonic drug treatment in conjunction with psychotherapy sessions designed to elicit the memories. Meditation may be prescribed depending on the willingness and ability of the subject. When administered concurrently with standard antidepressant drug treatment familiar to clinicians skilled in the art, a decrease in the typical delay of several weeks between antidepressant-treatment onset and the start of the therapeutic antidepressant effect is observed.

E) Schizophrenia

Brain mechanisms of learning and memory have been hypothesized to underlie delusions in schizophrenia (Eichenbaum and Bodkin Belief and Knowledge as Distinct Forms of Memory. In: Memory, Brain and Belief. Schacter D L and Scarry E, Eds. Cambridge, Mass.: Harvard University Press, 2000, pp. 176-207). Antimnemonic therapy is useful in the treatment of these symptoms. Patients diagnosed as suffering from schizophrenia receive antimnemonic drug treatment in conjunction with psychotherapy sessions or other verbal stimuli, clinical cue-exposure sessions and/or environmental cue exposure. Psychotherapy or meditation is preferably used to elicit memories if intrusive memories are reported by the patient. Cue presentation, either clinical or environmental is preferred if the patients symptoms are determined to be related to environmental stimuli. In one embodiment the patient is prescribed an antimnemonic drug and instructed to self-administer the drug immediately following the onset of symptoms.

Neuroleptics are both a standard treatment for schizophrenia, familiar to clinicians skilled in the art, and may serve as antimnemonic drugs. Thus, the schizophrenic's standard neuroleptic regimen may be incorporated into the antimnemonic therapy. These embodiments, which could involve other antimnemonic drugs in addition to the neuroleptic regimen, may decrease the typical delay of several weeks between neuroleptic-treatment onset and the start of the antipsychotic effect of neuroleptics.

F) Antisocial Personality Disorder (APD)

The significant environmental influence on antisocial behavior, as revealed in twin and adoption studies, may reflect peer group influences and parenting style (Rhee S H and Waldman I D, Genetic and environmental influences on antisocial behavior: A meta-analysis of twin and adoption studies. Psychological Bull. 128:490-529, 2002). Therefore, to the extent that peer and parental influences are recorded in memory, antimnemonic therapy benefits people with APD. Society at large may also benefit from an effective treatment for APD because this diagnosis is greatly over-represented in prisons. Surveys of 23,000 prisoners showed that about half of the males and one fifth of females had APD (Fazel S and Danesh J. Lancet 359:545-550, 2002).

Subjects with APD receive antimnemonic drug treatment together with cue sessions, field trips (if possible), and psychotherapy depending on whether subjects report more influences from environmental cues or troubling memories. Psychotherapy or meditation is preferably used to elicit memories if intrusive memories are reported by the patient. Cue presentation, either clinical or environmental is preferred if the patients symptoms are determined to be related to environmental stimuli. Homework sessions and meditation are prescribed depending on the willingness and ability of the subject.

What is claimed is:

1. A method of treating a behavioral disorder comprising:
a) presenting a cue associated with the disorder to a patient; and
b) administering an antimnemonic drug to the patient, in either order.

2. The method of claim 1, wherein steps a) and b) are repeated as needed to alleviate symptoms of the disorder.

3. The method of claim 1, wherein the cue is at least one of a visual, olfactory, aural, tactile, or gustatory cue.

4. The method of any of claim 1, wherein the cue is presented in a clinical environment.

5. The method of claim 1, wherein the cue is part of the patient's natural environment outside of the clinic.

6. The method of claim 1, wherein the behavioral disorder is a hypermemory disorder.

7. The method of claim 6, wherein the hypermemory disorder is selected from the group consisting of addiction, obsessive-compulsive disorder, Tourette's Syndrome, post-traumatic stress disorder (PTSD), bipolar disorder, depression, schizophrenia, anxiety disorders and personality disorders.

8. The method of claim 7, wherein the hypermemory disorder is addiction.

9. The method of claim 1, wherein the antimnemonic drug is selected from the group consisting of benzodiazepines, NMDA-receptor antagonists, glucocorticoid receptor antagonists, α2-adrenoceptor agonists, β-adrenoceptor antagonists, muscarinic cholinergic antagonists, protein kinase A inhibitors, protein kinase C inhibitors, calcium/calmodulin dependent kinase inhibitors, mitogen-activated protein kinase kinase inhibitors, cyclic adenosine monophosphate response element binding protein inhibitors, nitric oxide synthase inhibitors and GABA receptor agonists.

10. The method of claim 1, wherein the antimnemonic drug is selected from the group consisting of memantine, muscimol, clonidine, metoprolol, atropine, ecopipam, sulpiride, haloperidol, 7-nitroindazole, ibenztropine, scopolamine, propranolol, dextromethorphan, midazolam and lorazepam.

11. The method of claim 10, wherein the antimnemonic drug is memantine.

12. The method of claim 11, wherein the memantine is administered at a dose of between 0.1 and 5 mg/day.

13. The method of claim 1, wherein the antimnemonic drug is administered by a clinician.

14. The method of claim 1, wherein the antimnemonic drug is self-administered by the patient.

15. The method of claim 1, wherein the antimnemonic drug is administered within five hours following cue presentation.

16. The method of claim 15, wherein the antimnemonic drug is administered within thirty minutes following cue presentation.

17. The method of claim 1, wherein the antimnemonic drug is administered up to one hour prior to cue presentation.

18. A method of treating a behavioral disorder comprising:
a) reactivating a memory associated with the disorder in a patient; and
b) administering an antimnemonic drug to the patient, in either order.

19. The method of claim 18, wherein steps a) and b) are repeated as needed to alleviate symptoms or to maintain symptoms in an alleviated state.

20. The method of claim 18, wherein reactivation is triggered by exposure of the patient to a cue.

21. The method of claim 20, wherein the cue is at least one of a visual, olfactory, aural, tactile, or gustatory cue.

22. The method of claim 18, wherein reactivation is triggered by psychotherapy.

23. The method of claim 18, wherein reactivation is triggered by homework.

24. The method of any of claims 18, wherein reactivation occurs in a non-clinical environment.

25. The method of claim 18, wherein the behavioral disorder is a hypermemory disorder.

26. The method of claim 25, wherein the hypermemory disorder is a behavioral disorder included in the reward deficiency syndrome.

27. The method of claim 18, wherein the hypermemory disorder is addiction.

28. A method of treating addiction comprising:
a) exposing a patient to a cue or cues associated with the addiction;
b) administering an antimnemonic drug to the patient, in either order.

29. The method of claim 28, wherein the addiction is selected from the group consisting of addiction to drugs, gambling, food, sex, thrill-seeking, violence, political power, money-and computer technology.

30. The method of claim 28, wherein the addiction is addiction to drugs.

31. The method of claim 30, wherein the addiction to drugs is selected from the group consisting of addiction to alcohol, addiction to cocaine, addiction to nicotine, addiction to Cannabis, addiction to opiates and addiction to opiate derivatives

32. The method of claim 30, wherein exposing the patient to a cue comprises exposing the patient to a drug-related stimulus selected from the group consisting of audiotapes, videotapes, actors performing simulated drug administration rituals, drug paraphernalia and photographs of drug paraphernalia.

33. The method of claim 31, wherein the addiction is nicotine addiction.

34. The method of claim 33, wherein the cue comprises exposure to cigarette smoke.

35. The method of claim 31, wherein the addiction is addiction to alcohol.

36. The method of claim 36, wherein the cue comprises exposure to the smell of alcoholic beverages.

37. The method of claim 33, wherein the addiction is addiction to cocaine.

38. The method of claim 38, wherein the cue comprises exposure to the smell of burning baking soda.
